(19) United States
(12) Patent Application Publication
(10) Pub. No.: US 2009/0074709 A1
(43) Pub. Date: Mar. 19, 2009

(54) METHODS, DEVICES AND SYSTEMS FOR BIOCIDAL SURFACE ACTIVITY

(76) Inventors: RICHARD R. KOEPSEL, PITTSBURGH, PA (US); ALAN J. RUSSELL, GIBSONIA, PA (US); THANGAPPAN RAVIKUMAR, SACRAMENTO, CA (US)

Correspondence Address: BARTONY & HARE, LLP, 1806 FRICK BUILDING, 437 GRANT STREET, PITTSBURGH, PA, 15219-6101, US

(21) Appl. No.: 12/209,334

(22) Filed: Sep. 12, 2008

Related U.S. Application Data
(60) Provisional application No. 60/993,767, filed on Sep. 14, 2007.

(51) Int. Cl. A01N 43/40; A01N 37/06; A01N 43/36; C08F 118/02; C08F 226/06; C08F 20/52; C08F 20/44; C08F 22/10; C07C 69/52; C07D 211/20; C07C 255/08; C07C 211/62; C07C 233/01; C07D 213/20; A01P 1/00

(52) U.S. Cl. 424/78.32; 424/78.08; 424/78.35; 526/319; 526/265; 526/303.1; 526/341; 526/310; 526/321; 560/222; 546/248; 558/462; 564/288; 564/204; 546/347

(57) ABSTRACT

A method of applying a biocidal polymer to a surface including applying a solution of the biocidal polymer to the surface, wherein the biocidal polymer includes biocidal groups and the biocidal polymer is insoluble in water. The biocidal groups can be selected from the group consisting of quaternary salt groups or haloamino groups.

### CROSS-REFERENCE TO RELATED APPLICATION

This application claims benefit of U.S. Provisional Patent Application Ser. No. 60/993,767, filed Sep. 14, 2007, the disclosure of which is incorporated herein by reference.

### GOVERNMENTAL INTEREST

This invention was made with government support under grant DAAD19-01-0619 awarded by the DoD Multidisciplinary University Research Initiative. The government has certain rights in this invention.

### BACKGROUND OF THE INVENTION

The present invention relates to methods, devices and systems for biocidal surface activity and, particularly, to methods, devices and systems including surface-active antifungal groups such as polyquaternary amines and/or haloamines.

The following information is provided to assist the reader to understand the invention disclosed below and the environment in which it will typically be used. The terms used herein are not intended to be limited to any particular narrow interpretation unless clearly stated otherwise in this document. References set forth herein may facilitate understanding of the present invention or the background of the present invention. The disclosure of all references cited herein are incorporated by reference.

In a world where the use of soluble antimicrobial compounds leads to rapid emergence of resistant strains, there is a need for materials that can kill bacteria and fungi but remain bound to surfaces and are thus less likely to induce resistance. Cationic antimicrobials are especially well positioned to play a role in the development of self-disinfecting surfaces. See, for example, Gilbert, P.; Moore, L. E. J. Appl. Microbiol. 2005, 99, 703-715. The ability of such compounds to kill or inhibit a wide range of microorganisms enables the compounds to be used for a number of applications from hospital surfaces and medical devices to building materials and filtration devices.

Among the most commonly used of the cationic antimicrobials are the quaternary ammonium salts. Within this group of compounds, the polymeric quaternary amines show great promise in the realm of surface active compounds. Gilbert, P.; Moore, L. E. J. Appl. Microbiol. 2005, 99, 703-715. Since much of the recent investigations into the antimicrobial activity of polyquaternary amines has been directed toward activity against bacteria it is useful to briefly review growth of filamentous fungi as a contrast to single-celled bacteria.

Vegetative growth of filamentous fungi begins with the germination of spores. Spore germination leads to the formation of tubular hyphae which grow by apical extension and sub-apical branching. Macroscopically, the mycelium forms as a radially symmetric colony that expands at a constant rate from the site of spore germination. The hyphal mat, or mycelium, grows on the surface and, whenever possible, the sub-surface of the substrate. For the species of fungi commonly referred to as molds, reproduction is accomplished through the formation of spores which are disseminated primarily as aerosols.

The process of sporulation occurs in specialized structures which form on aerial hyphae which project through the water film that covers the substrate mycelium. The aerial structures must push through this water film to produce spores and release them into the atmosphere for distribution to new sites. The requirement for the water film over the substrate mycelium makes moist conditions ideal for fungal growth. During the sporulation process, the fungus must be able to overcome the surface tension of the water film and extend mycelia into the air. Prevention of mold growth in moist environments is a challenge for researchers as well as commercial workers and consumers. In addition to the extracellular enzymes the fungi release to degrade their substrata, they are known to produce a large number of other types of molecules. Many of these molecules, such as the mycotoxins, are toxic to other organisms and can be quite dangerous. These toxins are thought to be responsible for many of the symptoms associated with sick building syndrome.

Activity against bacteria does not insure activity against fungi. There are indications that synthetic polymeric quaternary amines, which are polycations, will have activity against fungi. Supporting the possibility that poly-quaternary amines will be effective at reducing fungal growth are recent reports that chitosan, a polycationic polymer of glucosamine, shows antifungal activity. Rhoades J & Roller S (2000) Applied Environ Microbiol. 66:80-86 and Plascencia-Jatomea M, Viniegra G, Olayo R, Castillo-Ortega M M, & Shirai K (2003). Macromolecular Biosciences. 3:582-586. The activity of this material is thought to be related to its polycationic nature. Several reports have suggested that surface bound polycations are capable of killing various microbes, including yeasts by disrupting the integrity of the cell membrane. Lee, S. B.; Koepsel, R. R.; Morley, S. W.; Matyjaszewski, K.; Sun, Y.; Russell, A. J. Biomacromolecules 2004, 5, 877-882; Lin, J.; Qiu, S.; Lewis, K.; Klibanov, A. M. Biotechnol. Bioeng. 2003, 83, 168-172; Milovic, N. M.; Wang, J.; Lewis, K.; Klibanov, A. M. Biotechnol, Bioeng. 2005, 90, 715-722; Waschinski, C. J.; Tiller, J. C. Biomacromolecules 2005, 6, 235-243.; Tiller J C, Liao C J, Lewis K, &. Klibanov A M (2001) Proc Nat Acd Sci USA. 98:5981-5985 and Kugler R., Bouloussa O, & Rondelez F. (2005). Microbiol. 151:1341-1348.

Several mechanistic hypothesizes have been put forward to explain the wide range of cells that are susceptible to polyquaternary amines including recruitment of membrane lipids into membrane blebs causing disruption of function and direct insertion of the polymer into the membrane. See, Gilbert, P.; Moore, L. E. J. Appl. Microbiol. 2005, 99, 703-715 and Lin, J.; Qiu, S.; Lewis, K.; Klibanov, A. M. Biotechnol. Bioeng. 2003, 83, 168-172. A somewhat simpler and perhaps more universal mechanism posits that the charge density of the surface induces an ion exchange, causing essential structural divalent ions to move out of the membrane resulting in a loss of membrane integrity. See Published PCT International Patent Application No. WO 2007/061954 and Kugler R., Bouloussa O, & Rondelez F. (2005). Microbiol. 151:1341-1348. Recently, preparations of quaternized poly(2-dimethylaminoethyl methacrylate) (poly-DMAEMA) have been synthesized as cationic surfactants, as polymer microspheres, and on glass and paper surfaces. All demonstrate high levels of antibacterial activity, and it is possible that the mechanism by which they kill cells is dependent upon the physical form that they take. See Lenoir, S.; Pagnoulle, C.; Detrembleur, C.; Galleni, M.; Jerome, R., J. Polymer Sci, Part A: Polym. Chem. 2006, 44, 1214-1224; Cheng, Z.; Zhu, X.; Shi, Z. L.; Neoh, K. G.; Kang, E. T. Ind. Eng Chem. Res. 2005, 44, 7098-7104; and Andersson M A, Nikulin M, Koljalg U, Andersson M C, Rainey F, Reijula K. Hintikka E-L, & Salkinoja-Salonen M. (1997) Applied Environ Microbiol. 63:387-393. Soluble polymers are more likely to be able to penetrate cell walls and membranes, whereas surface bound ones are more likely limited to a charge-transfer mechanism.

Although advances have been made in the development of biocidal surfaces, there remains a need within numerous industries for stable, surface-active, biocidal compositions, systems and methods of application.

### SUMMARY OF THE INVENTION

In general, the present invention provides methods, systems and compositions for biocidal surface. The surface-active biocidal compositions of the present invention are active against bacteria and fungi and can be applied either pre- or post-formation of an article or construction.

In one aspect, the present invention provides a method of applying a biocidal polymer to a surface including applying a solution of the biocidal polymer to the surface, wherein the biocidal polymer comprises biocidal groups and the biocidal polymer is insoluble in water. For example, the biocidal polymers can have a solubility in water of less than 50 μg/ml, less than 25 μg/ml, less than 10 μg/ml or even lower at, for example, room temperature or 25° C. In general, the solution is in a nonaqueous solvent. The polymer can be hydrophobic. The biocidal groups can, for example, be quaternary salt groups or haloamino groups. The haloamino groups can, for example, be halohydantoin groups.

In several embodiments, the biocidal polymer is formed via a process including a radical polymerization. For example, the biocidal polymer can be formed via a process including a controlled radical polymerization.

In a number of embodiments, the biocidal groups are the quaternary salt groups such as quaternary ammonium salt groups or quaternary phosphonium salt groups. Polymers including quaternary ammonium salt groups (as well as polymers including haloamino groups) can be formed from radically polymerizable monomers or compounds including at least one amino group. Radically polymerizable monomers including amino groups include, for example, 2-(dimethylamino)ethyl methacrylate), 4-vinyl pyridine, 2-vinyl pyridine, N-substituted acrylamides, N-acryloyl pyrrolidine, N-acryloyl piperidine, acryl-L-amino acid amides, acrylonitriles, methacrylonitriles vinyl acetates, 2-hydroxy ethyl methacrylate, p-chloromethyl styrene, and derivatives and substituted varieties of such monomers.

Quaternary ammonium salt groups can, for example, be formed by reaction of a compound of the formula R₈Q with amino groups on radically polymerizable monomers reacted to form the polymer or with amino groups on a precursor polymer formed from radically polymerizable monomers. R₈ can, for example, be an alkyl group of at least 8 carbon atoms, a fluorinated alkyl group of at least 8 carbon atoms or an aromatic group.

The terms “alkyl”, “aromatic” and other groups refer generally to both unsubstituted and substituted groups unless specified to the contrary. Unless otherwise specified, alkyl groups are hydrocarbon groups. Alkyl groups, and can be branched or unbranched, acyclic or cyclic. In fluoroalkyl groups, one or more of the hydrogen atoms of an alkyl group a substituted with fluorine. The above definition of an alkyl group and other definitions apply also when the group is a substituent on another group. The terms “aryl” or “aromatic” refers generally to substituted and unsubstituted phenyl groups, substituted and unsubstituted naphthyl aromatic groups, and substituted and unsubstituted pyridine groups. For example, p-toluenesulphonyl is a possible counter anion for ammonium salts.

Q can, for example be a halide (Cl, Br, F or I), CF₃SO₃ or CF₃CO₂. As used herein, the terms “halide”, “halogen” or “halo” refer to fluoro (F), chloro (C), bromo (Br) and iodo (I).

In several embodiments, R₈ is an alkyl group or a fluorinated alkyl group of at least 8 carbon atoms or at least 12 carbon atoms. Such alkyl groups (or substituted alkyl groups) can be branched or unbranched/linear, acyclic or cyclic. In a number of embodiments, the alkyl group is a C₈ to C₂₂ alkyl group.

In several embodiments, the quaternary ammonium salt groups are formed by reaction of a compound of the formula R₈Q with amino groups on radically polymerizable monomers which are subsequently reacted to form the biocidal polymer.

The biocidal polymer can, for example, include at least one repeat unit selected from the following formulae:

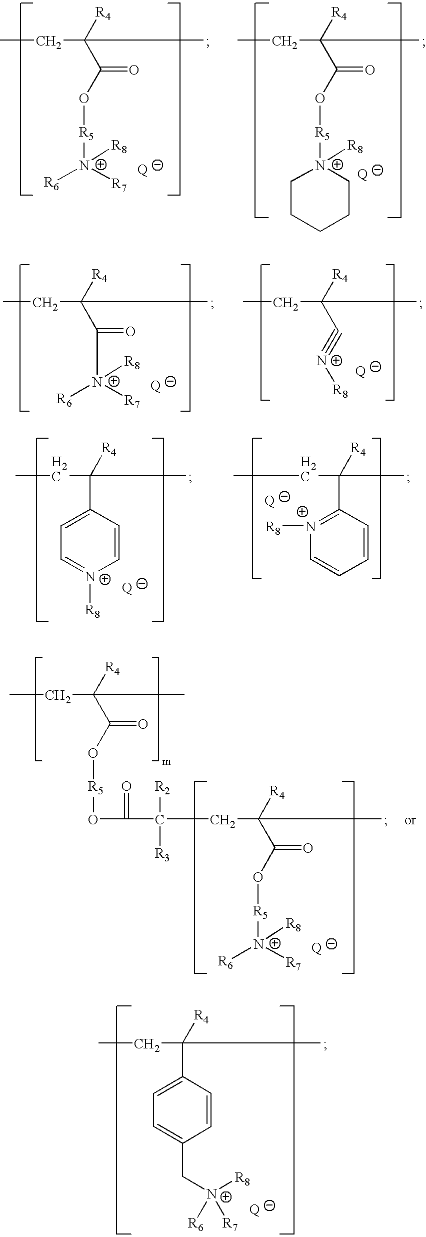

wherein R₂ and R₃ are, independently, H, CH₃, OOCC₂H₅ or CN, R₄ is H, CH₃, Cl or CN, R₅ is —(CH₂)ₙ— or —CH₂C(CH₃)₂CH₂—, wherein n is an integer from 1 to 6, R₆ and R₇ are, independently, a C₁-C₅ alkyl (for example, an isopropyl group), R₈ is an alkyl group of at least 8 carbons, a fluorinated alkyl group of at least 8 carbons or an aromatic group and Q is one of F, Cl, Br, I, CF₃SO₃ and CF₃CO₂.

In another aspect, the present invention provides a biocidal article formed by applying a solution of a biocidal polymer in a nonaqueous solvent to a surface of an article, wherein the biocidal polymer comprises biocidal groups and the polymer is insoluble in water.

In another aspect, the present invention provides a polymer that is the reaction product of a radical polymerization of monomers comprising at least one radically polymerizable monomer of the following formulae:

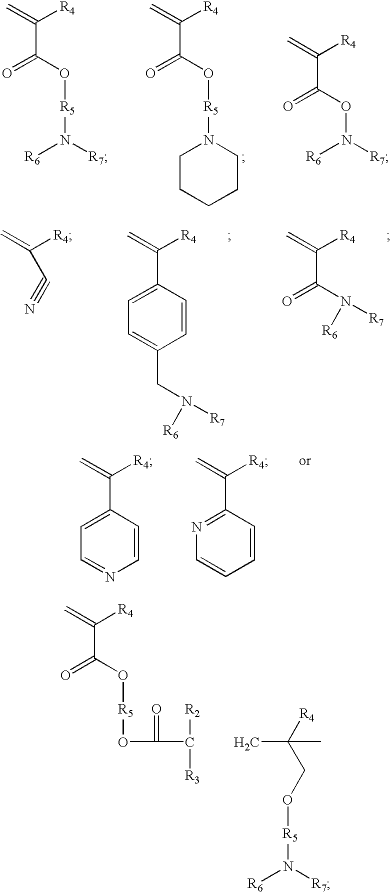

wherein an amino group of the radically polymerizable monomer is converted to a quaternary salt either before or after the radical polymerization by reaction with a compound of the formula R₈Q, wherein R₈ is an alkyl group of at least 8 carbon group, a fluorinated alkyl group of at least 8 carbon groups or an aromatic group and Q is F, Cl, Br, I, CF₃SO₃ or CF₃CO₂, and wherein R₂ and R₃ are, independently, H, CH₃, OOCC₂H₅ or CN, R₄ is H, CH₃, Cl or CN, R₅ is —(CH₂)ₙ— or —CH₂C(CH₃)₂CH₂—, wherein n is an integer from 1 to 6, and R₆ and R₇ are, independently, one of C₁-C₅ alkyl or isopropyl, such that the polymer is insoluble in water.

The radically polymerizable monomers can, for example, include at least one radically polymerizable monomer of the following formulae:

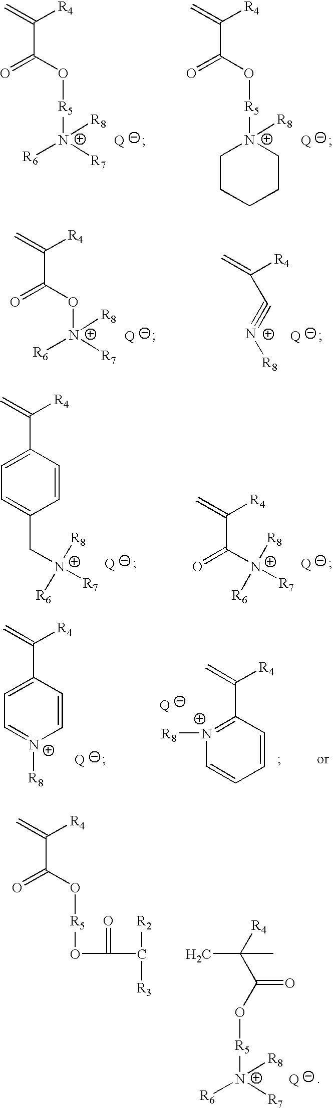

In several embodiments, the polymer is a homopolymer of at least one of the above radically polymerizable monomers. The radical polymerization can, for example, be a controlled radical polymerization. In a number of embodiments, R₈ is an alkyl group or fluoroalkyl group and Q is F, Cl, Br, or I. The alkyl group or the fluoroalkyl group can have at least 8 carbon atoms or at least 12 carbon atoms. R₈ can, for example, be chosen to result in a polymer that is insoluble in water as well as hydrophobic.

The radically polymerizable monomer can, for example, be

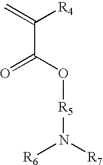

and R₈Q can, for example, be 1-bromododecane.

In still a further aspect, the present invention provides a compound having the formula:

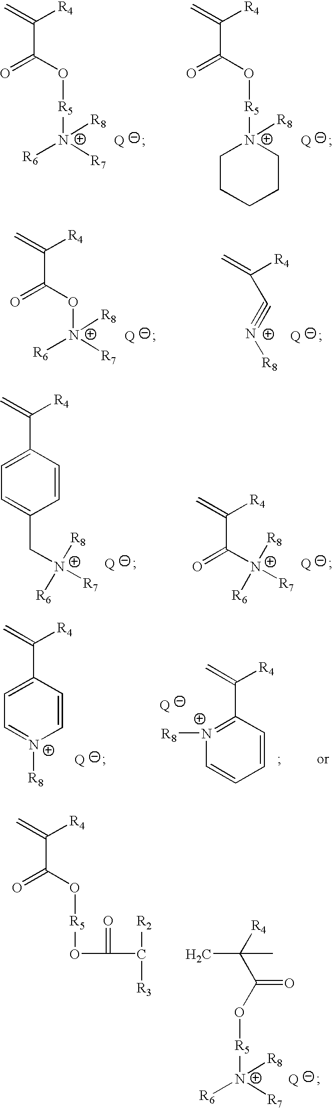

wherein R₂ and R₃ are, independently, H, CH₃, OOCC₂H₅ or CN, R₄ is H, CH₃, Cl or CN, R₅ is —(CH₂)ₙ— or —CH₂C(CH₃)₂CH₂—, n is an integer from 1 to 6, R₆ and R₇ are, independently, C₁-C₅ alkyl or isopropyl, R₈ is an alkyl group of at least 8 carbon atoms, a fluorinated alkyl group of at least 8 carbon atoms or an aromatic group; and Q is one of F, Cl, Br, I, CF₃SO₃ and CF₃CO₂.

The present invention, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates synthesis of two poly-quaternary amine derivatives of poly-2-dimethylaminoethyl methacrylate (poly-DMAEMA) by ATRP; wherein for PQA-C6, n=6; wherein for PQA-C12, n=12; and wherein m was about 100 for PAQ-C6 and about 220 for PAQ-C12.

FIG. 2A illustrates ripples formed in a well of a tissue culture plate when PQA-C12 is dried therein.

FIG. 2B illustrates magnification of the ripples formed by fluorescein containing PQA-C12.

FIG. 2C illustrates ripples in a well that was soaked with water for 48 hours.

FIG. 2D illustrates Growth of fungi in a well containing rippled PQA-C12.

FIG. 2E illustrates fluorescein staining of a PQA-C12 modified glass slide (fluorescein binds tightly to quaternary amines and fungi) after incubation with A. niger for 48 hours showing a complete absence of fungal mycelia.

FIG. 2F illustrates fluorescein staining of an unmodified glass slide after incubation with A. niger for 48 hours showing extensive fungal growth (wherein the contrast and brightness of the image are enhanced relative to FIG. 2E.)

FIG. 3A illustrates growth inhibition of A. niger by PQA-C6 measures at 24 hours.

FIG. 3B illustrates growth inhibition of A. niger by PQA-C12 measures at 24 hours.

FIG. 3C illustrates growth inhibition of A. niger by PQA-C6 measures at 48 hours.

FIG. 3D illustrates growth inhibition of A. niger by PQA-C12 measures at 48 hours.

FIG. 4 illustrates reduction in the number of viable colonies following incubation with PQAs wherein Aspergillus niger spores were treated by PQAs (1.5 mg/well) for 8 hours and aliquots were taken, diluted and plated on PDA agar to assess viability

FIG. 5 illustrates spore germination in the presence and absence of PQAs (Control/absence of PQAs (♦); PQA-C6 (▪); PQA-C12 (▴)).

FIG. 6 illustrates the number of spores recovered after one week incubation with and without PQA's.

FIG. 7A illustrates observation of growth in the absence of PQAs wherein the cultures were grown for 48 hours, harvested, stained with FungaLight™ and observed at 640X with white light using Nomarski optics.

FIG. 7B illustrates observation of growth in the absence of PQAs wherein the cultures were grown for 48 hours, harvested, stained with FungaLight™ and observed at 640X under fluorescence.

FIG. 7C illustrates observation of growth in the presence of PQA-C6 wherein the cultures were grown for 48 hours, harvested, stained with FungaLight™ and observed at 640X with white light using Nomarski optics.

FIG. 7D illustrates observation of growth in the presence of PQA-C6 wherein the cultures were grown for 48 hours, harvested, stained with FungaLight™ and observed at 640X under fluorescence.

FIG. 7E illustrates observation of growth in the presence of PQA-C12 wherein the cultures were grown for 48 hours, harvested, stained with FungaLight™ and observed at 640X with white light using Nomarski optics.

FIG. 7F illustrates observation of growth in the presence of PQA-C12 wherein the cultures were grown for 48 hours, harvested, stained with FungaLight™ and observed at 640X under fluorescence.

FIG. 8A illustrates wood coupons incubated with A. niger spores wherein the wood is untreated.

FIG. 8B illustrates wood coupons incubated with A. niger spores wherein the wood is treated with isopropyl alcohol.

FIG. 8C illustrates wood coupons incubated with A. niger spores wherein the wood is treated with PQA-C12.

FIG. 9 illustrates 1H NMR traces for the PQA-C6 and PQA-C12 polymers of FIG. 1.

FIG. 10 illustrates transmittance FTIR spectra obtained from PQA brushes quaternized with 1-bromohexane (a), with 1-bromononane (b) and with 1-bromododecane (c) on the double side polished silicon wafer.

FIG. 11 illustrates a representation of dry layer thickness of precursory PDMAEMA brush (right column) and after quaternization with different carbon number alkyl bromide (left) respectively.

### DETAILED DESCRIPTION OF THE INVENTION

To address the need for surface-active biocidal and, particularly, antifungal agents, we have synthesized representative polymeric quaternary amines via the representative controlled/living radical polymerization, atom transfer radical polymerization (ATRP). Previous work has shown that surface-bound and water soluble quaternary amine polymers have significant biocidal activity against the bacteria Escherichia coli and Bacillus subtilis. See, for example, Lee S B, Koepsel R R, Morley S W, Matyjaszewski K, Sun Y, & Russell A J (2004) Biomacromol. 5:877-882 However, as discussed above, activity against bacteria does not ensure activity against fungi. Desirable characteristics displayed by a surface-active antifungal agent include, but are not limited to, stability to transient immersion in water and prevention of fungi from growing on the treated surface.

As used herein, the terms “biocidal,” “biocidally active” or “antimicrobial” refer generally to an ability of a composition or group to inhibit the growth of, inhibit the reproduction of or kill microorganisms: such as, without limitation, spores and bacteria, fungi, mildew, mold, and algae.

Controlled variation of polymer physiochemical properties (polymer compositions, architectures, functionalities etc.) for the development of surface materials of the present invention with biocidal properties can, for example, be achieved using controlled/living radical polymerization or CRP processes. Atom transfer radical polymerization or ATRP, nitroxide mediated polymerization (NMP), reversible addition fragmentation chain transfer (RAFT) and catalytic chain transfer (CCT) are examples of controlled/living radical polymerization processes or CRP that provide versatile methods for controlled synthesis of polymers.

CRP processes differ, for example, in the type of group being transferred. For example, ATRP polymerizations typically involve the transfer of halogen groups. NMP polymerizations generally involve the transfer of stable free radical groups, such as nitroxyl groups. Details concerning nitroxide mediated polymerizations are described in, for example, in Chapter 10 of The Handbook of Radical Polymerization, K. Matyjaszewski & T. Davis, Ed., John Wiley & Sons, Hoboken, 2002. RAFT processes, described by Chiefari et al. in Macromolecules, 1998, 31, 5559, differ from nitroxide-mediated polymerizations in that the group that transfers is, for instance, a thiocarbonylthio group, although many other groups have been demonstrated. See, for example, McCormick and Lowe, Accounts of Chemical Research, 2004, 37, 312-325.

As used herein and in the appended claims, the singular forms “a,” “an”, and “the” include plural references unless the content clearly dictates otherwise. Thus, for example, reference to “a biocidal group” includes a plurality of such biocidal groups and equivalents thereof known to those skilled in the art, and so forth.

As used herein, the term “polymer” refers to a compound having multiple repeat units (or monomer units) and includes copolymers (including two, three, four or more monomers). Likewise, related terms such as “polymerization” and “polymerizable” include “copolymerization” and “copolymerizable”.

As use herein, the term “controlled” refers to the ability to produce a product having one or more properties which are reasonably close to their predicted value (presuming a particular initiator efficiency). For example, if one assumes 100% initiator efficiency, the molar ratio of monomer to initiator leads to a particular predicted molecular weight.

Similarly, one can “control” the polydispersity or molecular weight distribution by ensuring that the rate of deactivation is the same or greater than the initial rate of propagation. However, the importance of the relative deactivation/propagation rates decreases proportionally with increasing polymer chain length and/or increasing predicted molecular weight or degree of polymerization. Controlled radical polymerizations can produce polymers that, when grown from surfaces, have narrow molecular weight distributions, or polydispersities, such as less than or equal to 3, or in certain embodiments less than or equal to 2.0 or less than or equal to 1.5. In certain embodiments, molecular weight distributions of less than 1.2 can be achieved. Control of polymer properties in CRP, and in ATRP specifically, is discussed, for example, in Zhang, et al., Controlled/”Living” Radical Polymerization of 2-(Dimethylamino)ethyl Methacrylate, Macromolecules, 31, 5167-5169 (1998).

ATRP is one of the most robust CRP and a large number of monomers can be polymerized providing compositionally homogeneous well-defined polymers having predictable molecular weights, narrow molecular weight distribution, and high degree of end-functionalization. For this reason, ATRP is used in a number of representative studies of the present invention. Matyjaszewski and coworkers have produced a number of patents, patent applications and journal articles related to ATRP. See, for example, U.S. Pat. Nos. 5,763,546; 5,807,937; 5,789,487; 5,945,491; 6,111,022; 6,121,371; 6,124,411; 6,162,882; 6,624,262; 6,407,187; 6,512,060; 6,627,314; 6,790,919; 7,019,082; 7,049,373; 7,064,166; 7,157,530 and U.S. patent application Ser. Nos. 09/534,827; PCT/US04/09905; PCT/US05/007264; PCT/US05/007265; PCT/US06/33152 and PCT/US2006/048656,; Matyjaszewski, K., Ed. Controlled Radical Polymerization; ACS: Washington, D.C, 1998; ACS Symposium Series 685; Matyjaszewski, K., Ed. Controlled/Living Radical Polymerization. Progress in ATRP, NMP, and RAFT; ACS: Washington, D.C, 2000; ACS Symposium Series 768; Matyjaszewski, K., Davis, T. P., Eds. Handbook of Radical Polymerization; Wiley: Hoboken, 2002; Qiu, J.; Charleux, B.; Matyjaszewski, K. Prog. Polym. Sci. 2001, 26, 2083; and Davis, K. A.; Matyjaszewski, K. Adv. Polym. Sci. 2002, 159, 1, the disclosures of which are incorporated herein by reference. A used herein, “ATRP” or “atom transfer radical polymerization” refer generally to a controlled/living radical polymerization as, for example, described by Matyjaszewski in the Journal of Americal Chemical Society, vol. 117, page 5614 (1995), as well as in ACS Symposium Serves 768, and Handbook of Radical Polymerization, Wiley: Hobolcer 2002, Matyjaszewski, K and Davis, T, editors, the disclosure of which are incorporated by reference.

ATRP enables one to, for example, build block copolymers of tight dispersity with relative ease. Use of ATRP in the synthesis of a number of biocidal surface agents is described in Published PCT International Patent Application No. WO/2005/084159 and in Lee, S. B., Koepsel, R. R., Morley, S. W., Matyjaszewski, K., Sun, Y. and Russell, A. (2004), Biomacromolecules, 5, 877-882, the disclosures of which are incorporated herein by reference.

ATRP uses a monomer, an initiator with a transferable halogen, and a catalyst including a transition metal with a suitable ligand. An “ATRP initiator” is a chemical molecule, with a transferable halogen or pseudohalogen that can initiate chain growth. Fast initiation is desirable to obtain well-defined polymers with low polydispersities. A variety of initiators, typically alkyl halides, have been used successfully in ATRP. Many different types of halogenated compounds are potential initiators. Reversible atom transfer can occur between the transition metal complex and the growing radicals thereby reducing the free radical concentration and decreasing the probability of termination by radical coupling. ATRP can, for example, be used in many solvents to grow polymers from surfaces.

Many monomers have been successfully polymerized by CRP, including ATRP. See Handbook at Radical Polymerization, Matyjaszewski, K and Davis, T. P., John Wiley and Sons, Inc., Hoboken, New Jersey (2002), the disclosure of which is incorporated herein by reference. In general, vinyl monomers are used in ATRP. The synthesized polymers may be homopolymers, copolymers, block polymer, graft polymers, dendritic polymers, random copolymers, comb polymers, branched polymers, star polymers, hyperbranched polymers, polymeric brushes, as well as any other polymeric structure that allow access of biocidally active groups to the organism. The biocidally active group can be incorporated into the entire backbone, a single block, multiple blocks, or branches of the homopolymers or copolymer or in more than one part of the polymer.

A number of biocidally active agents can be incorporated into the biocidal surface agents of the present invention. Classes of known biocidal agents include, for example, quaternary cation-containing polymers (for example, polyquaternary ammonium ion-containing or phosphonium ion-containing polymers), haloamines (for example, halohydantoins) and porphyrin derivatives.

Polyquaternary ammonium or phosphonium ion-containing polymers derivatives are known to effectively kill cells and spores by disrupting membranes.

Haloamines such as chloramines are renewable bleaches that can oxidize and kill. Haloamines not only kill, but also release oxidants that disrupt bacteria, spores and, potentially more importantly, the debris that is released from destroyed cells. The haloamines-induced degradation of cell and spore debris can thereby induce self-renewal of surface immobilized materials of the present invention. Examples of biocidally active haloamines include halohydantoins. Polymers including hydantoin groups can, for example, be formed from radically polymerizable monomers including hydantoin groups. A number of hydantoins suitable for use in the present invention have the general formula:

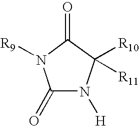

wherein one of R₉, R₁₀ and R₁₁ is a radically polymerizable group. R₉, R₁₀ and R₁₁ can for example be, independently (and differently) H, an alkyl group (for example, a C₁-C₃ alkyl group), or a group including an unsaturated group such as

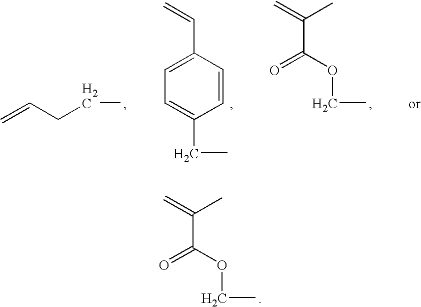

In addition to the above surface-active biocides, singlet delta oxygen (SDO) generating organic compounds can be incorporated within the matrix of the biocidal surface agents of the present invention. SDO can oxidize the biologicals (and contaminants) in a similar, but less aggressive manner, as TiO₂. SDO generators are already proven biocides. In the case of, for example, haloamines and porphyrin one can expect to kill spores and/or microbes and then degrade the debris.

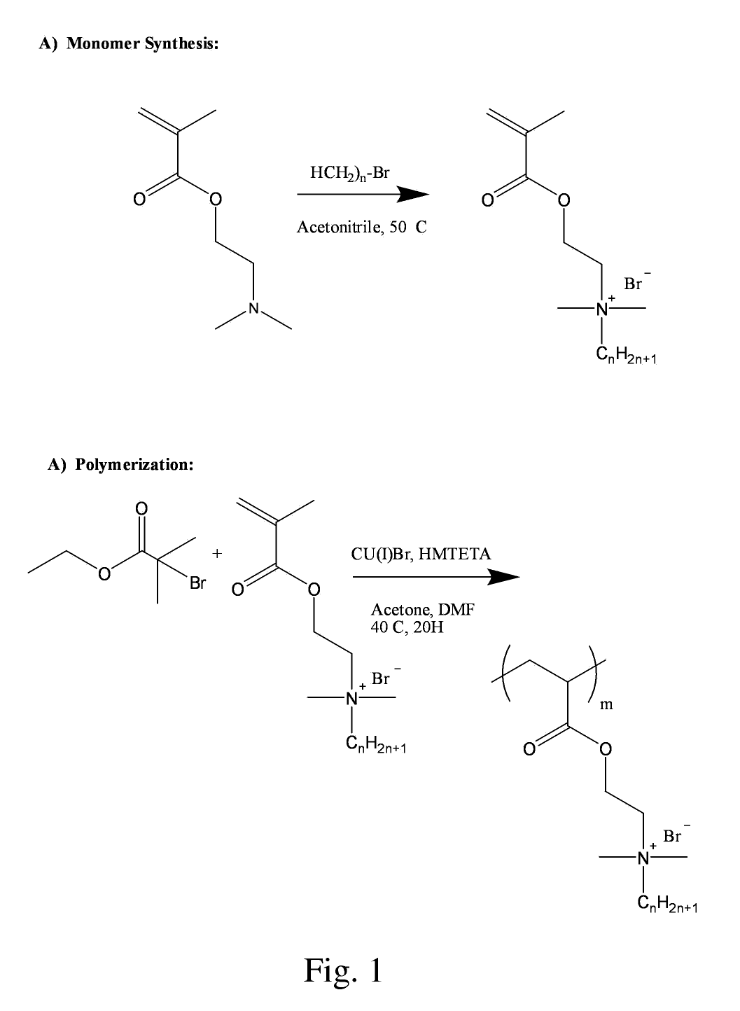

In representative studies of the present invention, we synthesized two derivatives of poly-2-dimethylaminoethyl methacrylate (poly-DMAEMA), PQA-C6 and PQA-C12, where the quaternary amino group has one substituent which is either a 6- or 12-carbon alkane, respectively. Referring to FIG. 1, n is 6 in the case of PAQ-C6, and n is 12 in the case of PQA-12. A major difference between the two polymers is water solubility. In that regard, PQA-C6 is highly water soluble, while PQA-C12 is virtually water insoluble. We tested these compounds for their ability to inhibit the growth of Aspergillus niger_or A. niger. We demonstrated that both PQA-C6 and PQA-C12 inhibit the growth of A. niger. PQA-C6 is solution-active, and PQA-C12 is particularly effective as a surface-active agent.

Synthesis and Characterization of the Quaternary Ammonium Polymers

FIG. 1 outlines the synthesis of the representative polymeric quaternary amines used in this study. The polymers were assembled from monomers synthesized by quaternization of dimethylaminoethylmethacrylate (DMAEMA) with 1-bromohexane or 1-bromododecane. In the studies of the present invention, the resultant representative monomers, MAQA-C6 and MAQA-C12, were then polymerized by atom transfer radical polymerization (ATRP). ATRP was chosen as a robust and representative controlled radical polymerization. Those skilled in the art appreciated that other radical polymerization and controlled radical polymerization techniques can be used to form the biocidal polymers of the present invention.

In general, quaternary amines (as well as haloamines) can be produced from any unsaturated, radically polymerizable, monomer containing a primary, secondary or tertiary nitrogen (or a functionality that can be converted into quaternary amine (or haloamine) either before after the polymerization reaction). Monomers comprising the biocidally active groups can, for example, be derived from monomers such as 2-(dimethylamino)ethyl methacrylate (DMAEMA) as described above, 4-vinyl pyridine, 2-vinyl pyridine, N-substituted acrylamides, N-acryloyl pyrrolidine, N-acryloyl piperidine, acryl-L-amino acid amides, acrylonitriles, methacrylonitriles vinyl acetates, 2-hydroxy ethyl methacrylate, p-chloromethyl styrene, and derivatives and substituted varieties of such monomers and other amine containing unsaturated monomer. As described further below, in a number of embodiments, precursor monomers and reactants to convert the amine groups thereof to quaternary ammonium salts are chosen so that the biocidal polymers of the present invention are insoluble in water.

In the studies of the present invention, the representative monomer DMAEMA was first quaternized and then polymerized via ATRP. Alternatively, the polyDMAEMA (or other amine-containing polymer) can be synthesized and subsequently quaternized. In the case that the monomer is first quaternized before polymerization, generally all possible precursor amine groups can be quaternized. In the case that a amine-containing monomers are first polymerized and then quaternized, however, it is likely that less than 100% of the amine groups of the polymer are quaternized. Thus, quaternization prior to polymerization can possibly lead to higher charge densities in the resultant polymer.

An advantage of ATRP (and other controlled radical polymerization techniques) for preparing polymers is that the molecular weight distribution of the obtained polymer is relatively narrow. The molecular weight and the molecular weight distribution of the polymers, PQA-C6 and PQA-C12, were estimated by GPC measurement as 35,000 (degree of polymerization, ca. 100) and 93,000 (degree of polymerization, ca. 220) with relatively narrow poly-dispersity indices of 1.25 and 1.35, respectively.

Solubility of PQA-C6 and PQA-C12.

Surface active biocides that could be applied to construction materials are preferably hydrophobic, stable to environmental stresses, and insoluble in water. As described above, we synthesized water- and organic-soluble PQAs and compared their ability to kill fungi on surface and in solution.

When PQA-C6 is dried onto the bottom of a well in a 24 well tissue culture plate it can be completely re-solubilized in 1 ml of water. On the other hand PQA-C12 deposited on the surface from a solution in isopropanol (100 μl) is insoluble in water. Table 1 shows the results of one method used to determine the relative solubilities of PQA-C6 and PQA-C12 after they had been dried onto polystyrene surfaces. In the experiments of Table 1 water was added to wells containing 1.5 mg of the PQAs and the concentration of the PQA in the water solution was determined after 24 and 48 hours. The results show that PQA-C6 is quickly solubilized, with most of the material in solution within 24 hours. By contrast, the PQA-C12 was still undetectable after soaking in water for 48 hours.

TABLE 1

| | % recover | % recover |
|---|---|---|
| Sample | at 24 hours | at 48 hours |
| PQA-C6 | 83* | 80 |
| PWA-C12 | 0* | 0 |

*The lower detection limit for both PQA-C6 and PQA-12 was 50 μg/ml

PQA-C12 Remains on the Surface After Immersion in Water

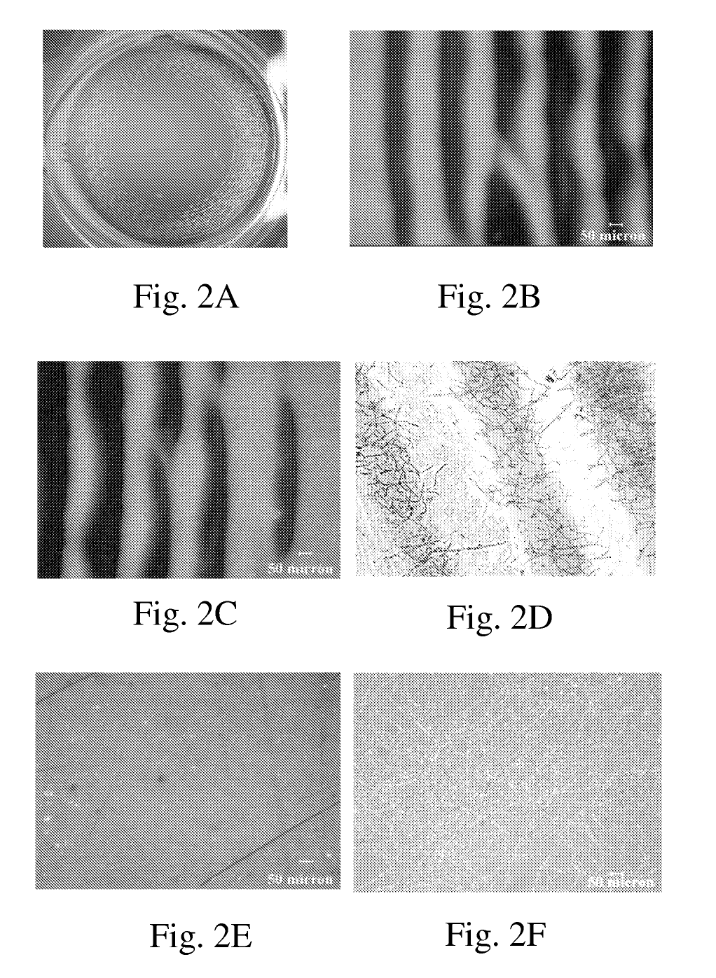

The surface-activity and non-leachability of PQA-C12 can be investigated elegantly because when PQA-C12 is dried onto the surface of the culture dish, the polymer self organizes as a series of concentric rings or ripples. An example of these ripples is shown in FIG. 2A. The ripples were a fortuitous artifact of the culture dish configuration and the drying procedure (under laminar flow) used in the present studies. Relatively, homogeneous surface coating can be readily formed with the biocidal polymers of the present invention. A variant of PQA-C12 which contains a small amount of fluorescein covalently attached to the polymer chain was synthesized and observation of ripples made with this fluorescent PQA-C12 shows that the PQA is concentrated in the ridges of the ripples (see FIG. 2B) with the troughs virtually free of the polymer. When wells prepared in this manner are soaked in water for 48 hours (see FIG. 2C) the ripples are not dissolved. The same experiment performed with PQA-C6 shows that the ripples disappear after as little as 30 minutes in water (data not shown). Interestingly when rippled wells are inoculated with fungi, the mycelia grow in the troughs between the ripples (see FIG. 2D). This observation further confirms that PQA-C12 remains on the surface after extensive washing, that it is indeed a surface active anti-fungal, and that it does not leach from the surface.

We have also grown PQA-C12 from the surface of glass slides using ATRP. In those experiments the covalent attachment of the polymer to the glass provides a uniform and non-leaching surface. Ellipsometry demonstrates that a 60-70 nm thick coating is routinely achieved by this method (data not shown). When fungi cultured on a PQA-C12 modified slide are compared to fungi on a plain glass surface the results are striking (see FIGS. 2E and 2F, respectively). Fungi grow rapidly on the untreated glass surface whereas the covalently coupled PQA-C12 prevents any fungal accumulation of the surface.

Dose Response Measurement of Inhibition of Fungal Growth by PQA's

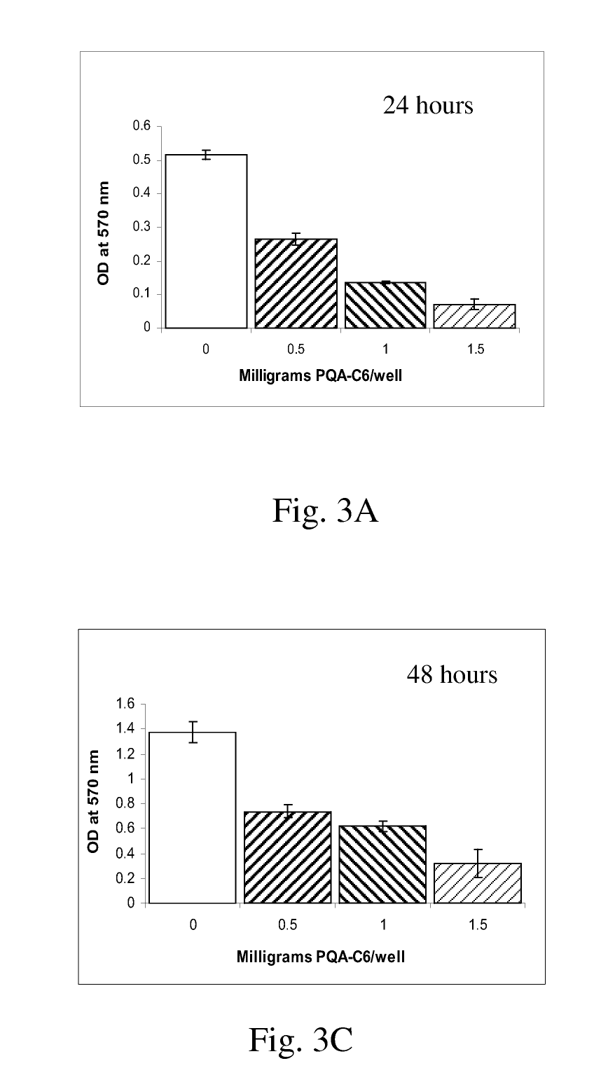

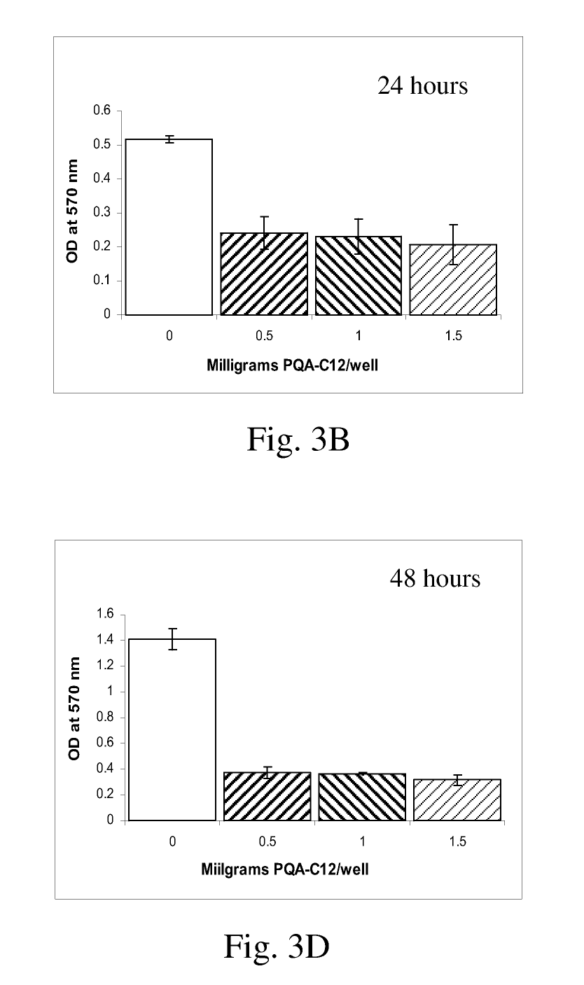

PQA-C6 and PQA-C12 were dissolved in isopropanol and applied to the wells of 6 well dishes at 0.5, 1.0 and 1.5 and dried as described above. Each well was then inoculated with 5000 A. niger spores in 1 ml of media. After 24 or 48 hours the relative mass of actively metabolizing fungi was determined with the MTT assay. FIGS. 3A through 3D shows the results of these experiments. The results of these inhibition studies illustrate that the two similar compounds display very different inhibition patterns. The PQA-C6 shows a classic dose response to the increasing amounts of polymer while the PQA-C12 response is flat across all of the concentrations at both time points tested. In the studies of FIG. 3A through 3D, A. niger was grown in wells of 24 well culture dishes containing PQA at various concentrations. The extent of growth was measured at 24 hours (see FIGS. 3A and 3B) and 48 hours (see FIGS. 3C and 3D) by MTT reduction. Results are reported as OD 570 (optical density at 570 nm) of the extracted formazan and are the average of 6 determinations.

Without limitation to any mechanism, the above result can be explained by again considering differences in the solubility of PQA-C6 and PQA-C12 in water. Since PQA-C6 is soluble in water, it will have complete access to the growing fungi throughout the media. Conversely, since PQA-C12 is insoluble, only the fungi that come in contact with the bottom of the well are killed. This solubility difference explains the flat dose-response of PQA-C12 because the effects would be more dependent on the surface area of the well than on the concentration of the polymer. In the PQA-C12 wells, the growing fungi are visible as a floating mat of hyphae on the surface of the media. Because PQA-C12 does not diffuse from the surface of the plate, the fungus on the surface of the media escapes the lethal activity of PQA-C12.

The Effect of PQAs on the Number of Colony Forming Units

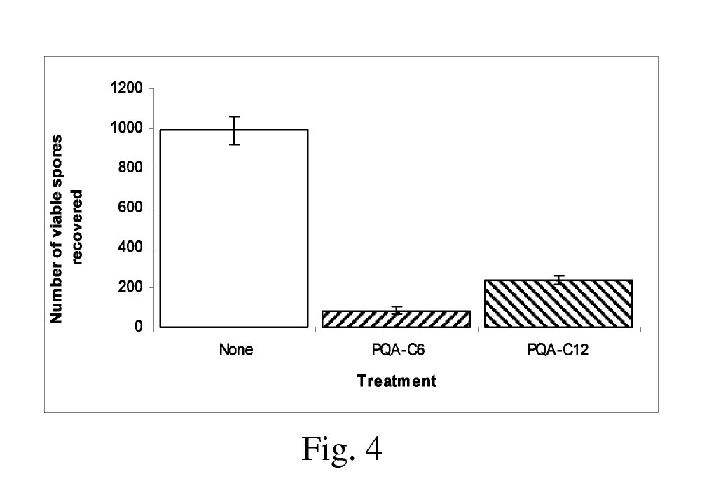

To confirm that the viability reduction was a result of reduced growth and not inhibition of oxidative metabolism, the number of viable fungal colonies was determined. In these experiments 1000 spores were incubated in wells with 1 ml of media with or without PQAs at 1.5 mg/well. After 8 hours incubation, aliquots were spread on potato dextrose agar plates to determine viable counts. As can be seen in FIG. 4, the PQAs dramatically reduced the number of colonies that appeared. Interestingly, the relative reduction in colony forming units was similar to the reduction measured by the MTT assay for both compounds in the longer term experiments.

Since 8 hour incubations approximate the time required for the added spores to undergo germination, the results in FIG. 4 could be interpreted as representing either a reduction in the viability of hyphae or as interference at the level of the germination of the fungal spores. Further experiments were conducted to differentiate between these possibilities.

PQAs Reduce the Rate of Germination but do not Reduce the Extent of Germination.

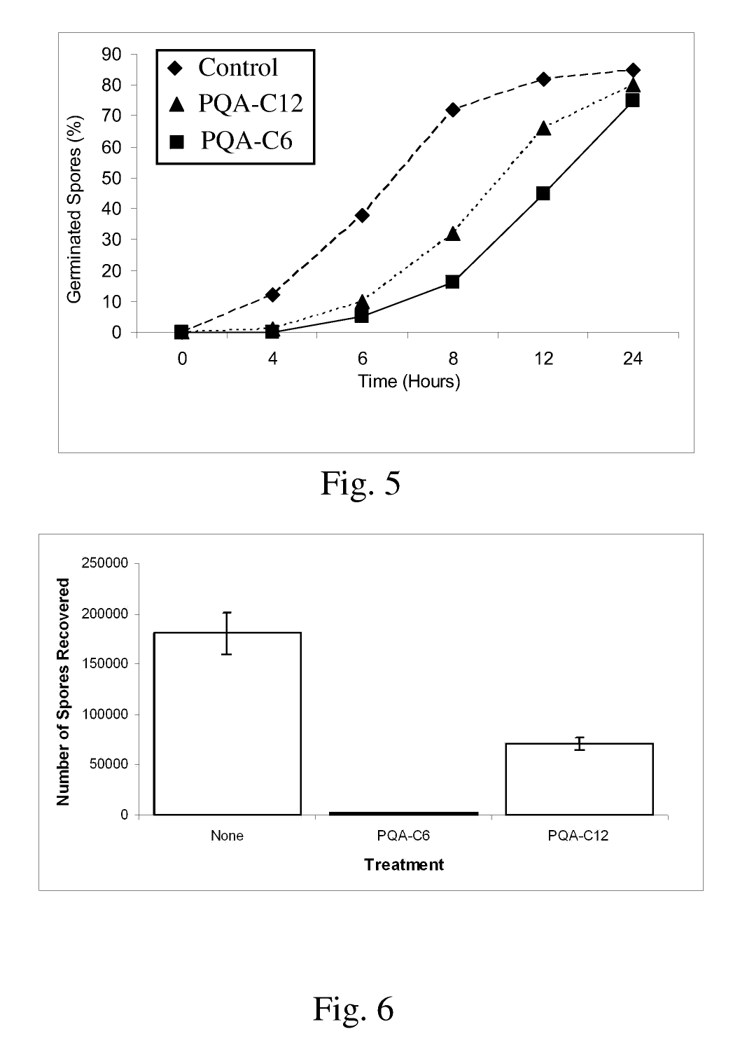

To test whether PQAs were acting as sporicides, we inoculated plates with spores as above, removed aliquots at various time points, and visually counted the number of germinated spores. The results of these observations are set forth in FIG. 5. In the studies of FIG. 5, spores in media were inoculated into wells and incubated at 30° C. At various times, samples were removed and loaded into a hemocytometer. The proportion of spores with visible germ tubes was determined for each sample at each time point. For spores incubated without PQAs, nearly 75% were germinated by 8 hours of incubation and >90% were germinated by 24 hours. Spores in the presence of PQA-C6 and PQA-C12 were only 15 and 30% germinated at 8 hours but were nearly 90% germinated by 24 hours. While the 8 hour results agree with the viable count assay (FIG. 5) they do not account for the fact that germination was completely recovered by 24 hours. Indeed, the results in FIG. 4 show that all of the spores used in the test were recovered as colonies in the controls even though only 70% had germinated within 8 hours. The spores from the PQA-treated wells would presumably complete germination on the agar plates after removal from the PQAs since they do so even in the presence of the PQAs.

A further possibility for the action of the PQAs is that they are killing the germinating spores before they can form a viable mycelium. Direct observation can not differentiate between a viable germ tube and a dead one. However, specific dyes are available to differentiate living versus dead fungal cells and spores. Aliquots from the 12-hour germination sample and the 24-hour PQA treated germination samples were stained with FungaLight™ (available from Invitrogen of Carlsbad, Calif.) to differentiate live from dead spores with or without germ tubes. Ten full field images were captured for each condition and the average number of live (green fluorescent) and dead (red fluorescent) spores was determined (see Table 2). No difference was found between the control surfaces and the PQA surfaces with respect to the number of viable germinated spores.

TABLE 2

| | | |
|---|---|---|
| Number of spores | | |
| per microscopic field | Viable Spores | Dead Spores |

| | | | |
|---|---|---|---|
| Control | 27.2 ± 1.8 | 24.0 ± 1.2 | 3.0 ± 1.2 |
| PQA-C6 treated | 26.0 ± 2.8 | 23.0 ± 2.6 | 3.0 ± 1.4 |
| PQA-C12 treated | 25.6 ± 2.8 | 22.6 ± 2.3 | 3.0 ± 1.2 |

Without limitation to any mechanism, it seems that the PQAs are not inhibiting the germination or the emergence of the germ tube; and that the growth inhibition must occur at a later stage of fungal development.

Sporulation of PQA Treated Aspergillus

As noted above, mats of fungal growth could be seen floating above the PQA-C12 surfaces. The possibility that a portion of the mycelia might survive to produce spores was tested. A. niger was grown in plates containing 1.5 mg/well of the two PQAs. After one week of growth the entire contents of the wells were harvested and the number of spores was determined. The results are shown in FIG. 6. Again the relative number of spores produced approximates the results from the MTT assays (FIG. 3). When all of the foregoing results are considered, the apparent difference in activities between the two PQAs can be attributed to the solubilities of the compounds and that PQA-C12 remains on the surface of the dish.

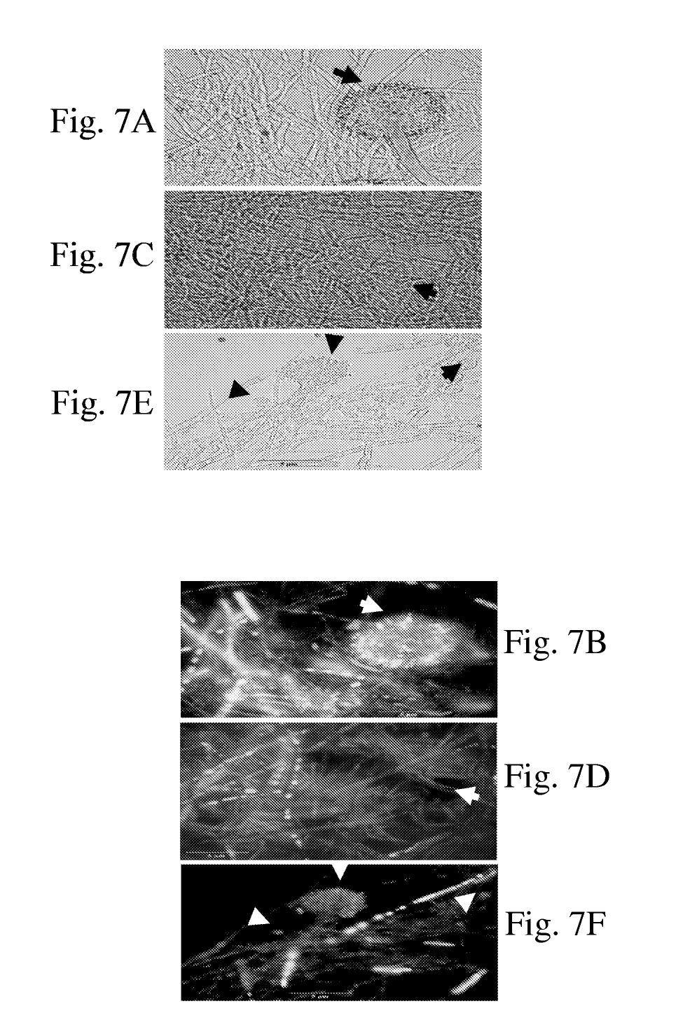

Because germination goes to completion with both PQAs, the FungaLight™ stain was used to test the activity of the compounds against mycelia. Observation of 48 hour cultures growing in wells containing PQAs shows the presence of some mycelia. These were harvested and stained with the FungaLight™ stains (FIGS. 7A through 7F). FIGS. 7A and 7B illustrate observation of growth in the absence of PQAs wherein the cultures were grown for 48 hours, harvested, stained with FungaLight™ and observed at 640X with white light using Nomarski optics and at 640X under fluorescence, respectively. FIGS. 7C and 7D illustrate observation of growth in the presence of PQA-C6 wherein the cultures were grown for 48 hours, harvested, stained with FungaLight™ and observed at 640X with white light using Nomarski optics and at 640X under fluorescence, respectively. FIGS. 7E and 7F illustrate observation of growth in the presence of PQA-C12 wherein the cultures were grown for 48 hours, harvested, stained with FungaLight™ and observed at 640X with white light using Nomarski optics and at 640X under fluorescence, respectively. With the FungaLight™, the live cells fluoresce green and the dead cells fluoresce red under fluorescence. However, grayscale images are set forth in FIGS. 7B, 7D and 7E. In the control culture, there is abundant mycelial growth and the formation of conidiophores and very few dead cells. When grown in the presence of the water soluble PQA-C6, the very few mycelia that can be found show an overwhelming abundance of dead cells. The situation is different with surface-bound PQA-C12. In this case there is a mixture of live and dead mycelia and the presence of some conidiophores. In the example shown, the conidiophore and most of the attendant spores appear to be dead but other examples show some viable spores (not shown).

In summary, both PQA-C6 and PQA-C12 significantly inhibit fungal growth; PQA-C6 is soluble in water, whereas PQA-C12 is not soluble in aqueous solution and acts as a non-diffusible antifungal coating. The PQAs have the ability to kill fungal mycelia but did not inhibit germination of spores. The PQAs appear to kill fungus at the more vulnerable mycelial stages of the life cycle of the fungus.

Without limitation to any mechanism, the insolubility of PQA-C12 indicates that an ion exchange mechanism is responsible for cell kill. For the polymer to penetrate the fungal cell wall and penetrate the membrane, it would need to be configured in such a way that the polymer chains extended a considerable distance from the surface. The low solubility of PQA-C12 suggest that a considerable chain extension is unlikely in an aqueous environment. The polymer may be in a compact form near the surface on which it is coated and would not likely be able to “see” the cell membrane. The polycationic nature of the polymer, however, persists in whatever architecture the polymer obtained and that this would be the active factor in the cell kill.

PQA-C12 Inhibits Fungal Growth on Wood

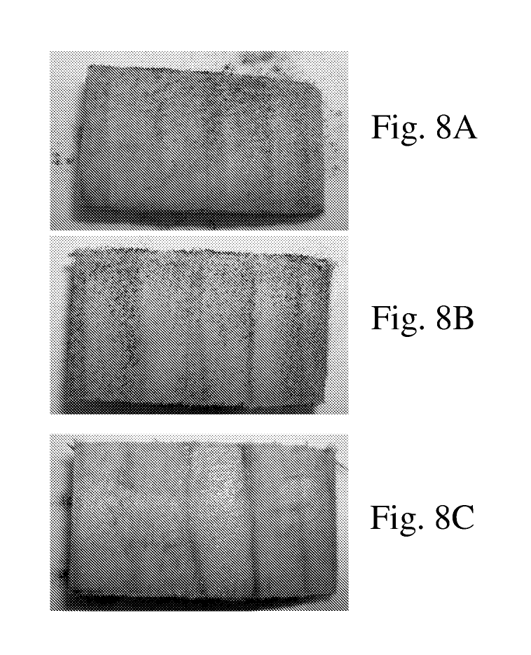

To illustrate the broad applicability of biocidal polymers of the present invention to a variety of surface, in several studies we painted the surface of wood coupons with PQA-C12 (1.5 mg/ml dissolved in isopropanol) or with isopropanol alone The coupons were placed in the wells of a 6-well culture dish with 1 ml potato dextrose broth and inoculated with 5000 A. niger spores. After 4 days in culture the coupons were photographed (see FIGS. 8A through 8C). Numerous small fungal colonies can be seen on the control (FIG. 8A) and isopropanol treated (FIG. 8B) coupons while the PQA-C12 coupon (FIG. 8C) remains free from fungal growth.

PQA-C12 is thus a surface-active antifungal agent that effectively prevents the proliferation of fungal mycelia. PQA-C12 is sufficiently hydrophobic that once it adsorbs to a surface, water will not remove it. In addition to a facile solution based synthesis, PQA-C12 can be grown directly from a surface, thereby imparting antifungal properties to that surface.

The biocidal polymers of the present invention can readily be applied from solution to a variety of surfaces (including, for example, polymeric surfaces, wood surfaces, metal surfaces, glass surfaces, ceramic surfaces and other surfaces) to produce a wide variety of biocidal (including antifungal) articles.

Experimental Methods and Results

Materials for Polymer Synthesis

N,N-dimethylaminoethyl methacrylate (DMAEMA), ethyl 2-bromoisobutyrate, 1,1,4,7,10,10-hexamethyltriethylenetetramine (HMTETA), copper (I) bromide (CuBr), 1-bromohexane, 1-bromododecane, 2-bromo-2-methylproionic acid bromide, acetone, acetonitrile, chloroform, methanol and N,N-dimethylformamide (DMF) were purchased from Sigma-Aldrich Chemical Co.

Measurement

¹H-NMR spectrum was recorded on a Bruker Avance (300 MHz) spectrometer in DMSO-D₆ and CDCl₃. Routine Fourier transform infrared (FT-IR) spectra were obtained with ATI Mattson Infinity series FT-IR spectrometer. Melting points (mp) were measured with a Laboratory Devices Mel-Temp. Number average molecular weights (Mₙ) and the distributions (M_{w}/Mₙ) were estimated by gel permeation chromatography (GPC) on a Waters 600E Series with a data processor, equipped with three polystyrene columns (Waters styragel HR1, HR2 and HR4), using DMF with LiBr (50 mM) as an eluent at a flow rate of 1.0 mL/min.

Monomer Synthesis

1-bromohexane (21.5 mL, 152.7 mmol) or 1-bromododecane (35 mL, 133.7 mmol) were added to a solution of DMAEMA (21.4 mL, 127.2 mmol) in acetonitrile (100 mL)/chloroform (50 mL), and stirred at 40° C. overnight. The resulting residues were precipitated into diethyl ether and filtered. The filtrates were dried in vacuo and analyzed. MAQAC₆; yield 37.8 g (92%), mp 85˜88° C., ¹H NMR (300 MHz, DMSO-d₆) δ 0.87 (t, 3 H, J=6.6 Hz, N⁺(CH₃)₂(CH₂)₅CH₃), 1.29 (broad m, 6 H, N⁺(CH₃)₂CH₂CH₂ (CH₂)₃CH₃), 1.69 (m, 2 H, N⁺(CH₃)₂CH₂CH₂ (CH₂)₃CH₃), 1.91 (s, 3 H, CH₂═C(CH₃)), 3.15 (s, 6 H, N⁺(CH₃)₂(CH₂)₅CH₃), 3.43 (m, 2 H, N⁺(CH₃)₂CH₂(CH₂)₄CH₃), 3.78 (m, 2 H, OCH₂CH₂N⁺(CH₃)₂(CH₂)₅CH₃), 4.64 (m, 2 H, OCH₂CH₂N⁻(CH₃)₂(CH₂)₅CH₃), 5.76 and 6.08 (s, 2 H, CH₂═C(CH₃)) ppm. IR (KBr) 3421, 3003, 2955, 2925, 2859, 1721, 1636, 1464, 1318, 1296, 1158, 957, 929, 809, 650 cm⁻¹. MAQAC₁₂; yield 44.1 g (86%), mp 80˜83° C., ¹H NMR (300 MHz, CDCl₃) δ 0.86 (t, 3 H, J=6.6 Hz, N⁺(CH₃)₂(CH₂)₁₁CH₃), 1.30 (broad m, 18 H, N⁺(CH₃)₂CH₂CH₂ (CH₂)₉CH₃), 1.74 (m, 2 H, N⁺(CH₃)₂CH₂CH₂ (CH₂)₉CH₃), 1.93 (s, 3 H, CH₂═C(CH₃)), 3.50 (s, 6 H, N⁺(CH₃)₂(CH₂)₁₁CH₃), 3.63 (m, 2 H, N⁺(CH₃)₂CH₂(CH₂)₁₀CH₃), 4.15 (m, 2 H, OCH₂CH₂N⁺(CH₃)₂(CH₂)₁₁CH₃), 4.64 (m, 2 H, OCH₂CH₂N⁺(CH₃)₂(CH₂)₁₁CH₃), 5.65 and 6.12 (s, 2 H, CH₂═C(CH₃)) ppm. IR (KBr) 3421, 3003, 2955, 2925, 2859, 1721, 1636, 1464, 1318, 1296, 1158, 957, 929, 809, 650 cm⁻¹.

Polymerizations.

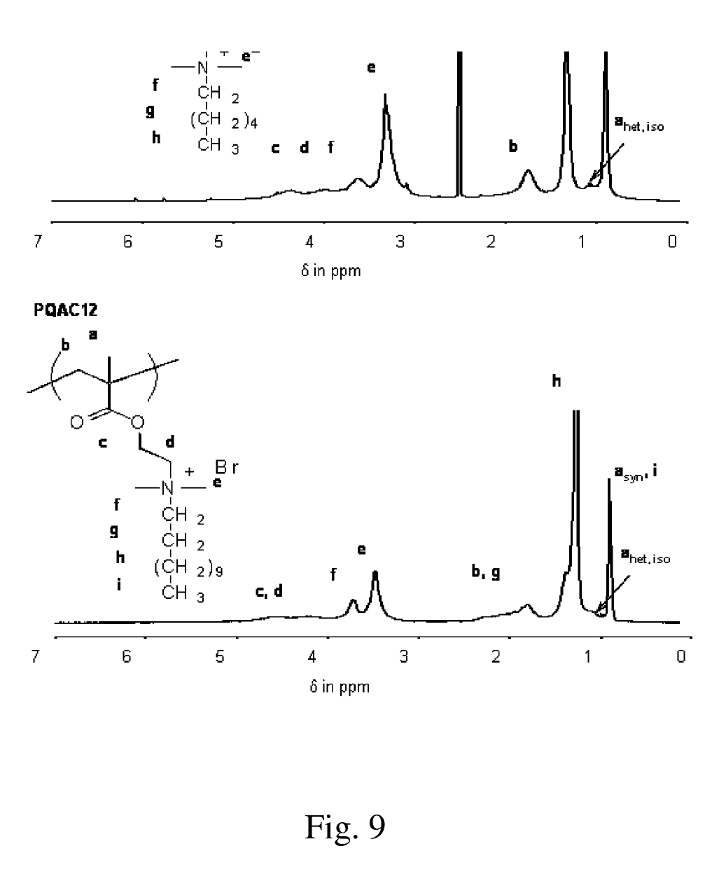

The monomer MAQAC₆ (3.2 g, 10.0 mmol) or MAQAC₁₂ (4.1 g, 10.0 mmol) was placed in a polymerization tube and covered with 2-bromoisobutyrate (14 μL, 0.1 mmol) as an initiator, and HMTETA (46 mL, 0.2 mmol) as the ligand in a solvent of acetone (35 mL)/DMF (5 mL). The monomer solutions were degassed by five freeze-pump-thaw cycles and then CuBr was added (28.7 mg, 0.2 mmol) as a catalyst. The resulting mixture was heated to 40° C. for 20 h. prior to dilution with acetone (10 mL). The polymerization solution was passed through a basic alumina column to remove the CuBr, and then precipitated in hexane. The resulting polymer was dried in vacuo and analyzed. PQAC₆: yield 2.8 g (88%), Mₙ; 35,000 g/mol and the distributions (M_{w}/Mₙ) 1.25, ¹H NMR (300 MHz, DMSO-d₃) δ 0.73 and 1.07 (broad s, total 6 H, —CH₂C(CH₃)— and N⁺(CH₃)₂(CH₂)₅CH₃), 1.31 (broad s, 8 H, N⁺(CH₃)₂CH₂(CH₂)₄CH₃), 1.71 (broad s, 2 H, —CH₂C(CH₃)—), 3.29 (broad s, 6 H, N⁺(CH₃)₂(CH₂)₅CH₃), 3.61 (broad s, 2 H, N⁺(CH₃)₂CH₂(CH₂)₄CH₃), 4.00 and 4.41 (broad s, 2 H and 2 H, OCH₂CH₂N⁺(CH₃)₂(CH₂)₅CH₃) ppm. IR (KBr) 3343, 2956, 2927, 1725, 1632, 1485, 1266, 1235, 1151, 1057, 961, 752 cm⁻¹. PQAC₁₂: yield 3.1 g (76%), Mₙ; 93,000 g/mol and the distributions (M_{w}/Mₙ) 1.35, ¹H NMR (300 MHz, CDCl₃) δ 0.84, 1.23, and 1.75 (broad s, total 26 H, —CH₂C(CH₃)— and N⁺(CH₃)₂CH₂(CH₂)₁₀CH₃), 2.26 (broad s, 2 H, N⁺(CH₃)₂CH₂CH₂(CH₂)₉CH₃), 3.30 (broad s, 6 H, N⁺(CH₃)₂(CH₂)₁₁CH₃), 3.67 (broad s, 2 H, N⁺(CH₃)₂CH₂(CH₂)₁₀CH₃), 4.02 and 4.55 (broad s, 2 H and 2 H, OCH₂CH₂N⁺(CH₃)₂(CH₂)₁₀CH₃) ppm. IR (KBr) 3432, 3005, 2955, 2926, 2860, 1725, 1639, 1475, 1266, 1235, 1151, 1050, 961, 752 cm⁻¹. FIG. 9 shows the 1H NMR traces for the polymers.

Synthesis of Covalently Attached Quaternary Ammonium Polymer Brush on Planar Glass Slides:

Synthesis of 3-(2-bromoisobutyroyl)-animopropyltrimethoxysilane. Allylamine (7.5 mL, 100 mmol), triethylamine (21 mL, 150 mmol) and dichloromethane (200 mL), was slowly added at 0° C. to a solution of 2-bromo-2-methylproionic acid bromide (13.6 mL, 110 mmol) in dichloromethane (50 ml), Next the mixture was stirred for 4 h at room temperature was and then washed with water followed sequentially by, a saturated aqueous solution of NaHCO₃, 0.5 M HCl, and a saturated aqueous solution of NaCl. The organic layer was dried over anhydrous MgSO₄, filtered, and concentrated by rotary evaporation. A solution of the allylic compound (5 mL, 24.2 mmol) and toluene (20 mL) was mixed with trimethoxysilane (7.7 mL, 60.5 mmol) and Pt/C (10% Pt, 100 mg), then the mixture was stirred at 60° C. overnight. Toluene and excess trimethoxysilane were removed by evaporation in vacuo. The crude residue was used for initiator immobilization after the Pt/C had been removed by filtration.

Initiator immobilization of on planar glass slides. Planar glass slides were placed into the initiator solution of 3-(2-bromoisobutyroyl)-animopropyltrimethoxysilane (100 μL), triethylamine (100 μL) and toluene (100 mL) at 80° C. for 1 hr. The treated slides were rinsed sequentially with toluene, methanol, and acetone.

“Surface-initiated” ATRP of DMAEMA on slides. The initiator modified slides were placed in a polymerization tube and covered with DMAEMA (8.4 mL), HMTETA (68 μL) and acetone (50 mL). The monomer solution was degassed by five freeze-pump-thaw cycles and Cu(I)Br (36 mg) was added. The mixture was heated to 40° C. and incubated for 20 h. The slides were then extracted in acetone for at least 6 h in a shaker to remove free polymer from the layer followed by a final rinse with methanol and then acetone.

Quaternization of Poly(DMAEMA) brush on glass surfaces with 1-bromododecane. Acetonitrile (50 mL), chloroform (10 mL), and 1-bromododecane (5 mL) were added to glass slides that had been modified with ester polymer brushes and incubated at 55° C. overnight. The glass slides were then extracted in chloroform, and rinsed with methanol and then with acetone.

Solubility Test of PQAs by Gel Permeation Chromatography

Stock solutions of PQA-C6 and PQA-C12 (50 mg/ml in isopropanol) were applied to the wells of 24 well tissue culture dishes (Fisher, Pittsburgh, Pa.) at 1.5 mg/well. The tissue culture dishes with the PQAs were dried completely under a laminar flow hood for 1-2 h. Residual solvent was further removed by keeping the dishes overnight in a vacuum desiccator. For each well, one ml of sterile distilled water was added and the plates were incubated at room temperature for 24 and 48 hours. At the designated time the entire volume was removed from the well, passed through a PTFE filter (0.2 μm) (Whatman, UK) and analyzed by gel permeation chromatography with a Waters model 2414 HPLC (Waters, Milford Mass.). Standard curves were generated using measured concentrations of the PQAs.

Fungal Growth

Aspergillus niger ATCC 9642 (American Type Culture Collection, Manassas Va.) was cultured in Potato Dextrose Broth (PDB) or Potato Dextrose Agar (PDA) (Difco, Detroit, Mich.). A. niger was cultured on PDA for 3-4 days to allow sporulation. Spores were collected by rubbing a sterile cotton swab gently on the surface of the plate followed by immersion in 1 ml solution of sterile distilled water with 10% Tween 20 (Sigma Chemical Co., St. Louis Mo.). The number of spores was estimated with a hemocytometer and stocks of 1-3×10⁶ spores/ml were prepared.

Fungal Growth Inhibition Studies

PQA C-6 or PQA C-12 (1.5, 1.0 or 0.5 mg) was coated on 24 well tissue culture plates as described above and dried under vacuum. Control wells were treated with IPA without added PQA. Depending on the experiment, each well was inoculated with between 10³ and 10⁴ Aspergillus niger spores and the plates were incubated at 30° C. for various times.

Antifungal activity was assessed by the MTT assay as described previously. Freimoser F M, Jakob C A, Aebi M, & Tuor U. (1999) Applied and Environ Microbiol. 65:3727-3729 and Kawada K, Yonei T, Ueoka H, Kiura K, Tabata M, Takigawa N, Harada M, & Tanimoto M (2002) Acta Medica Okayama. 56:129-134. Briefly, growth was assessed by the reduction of the tetrazolium dye 3-(4,5-dimethyl-2-thiazolyl)-2,5-diphenyl-2H-tetrazolium bromide (MTT) (Sigma, St. Louis Mo.). After different time points (usually 24, 48, and 72 h), the medium was removed from each well and 0.1 mL of fresh PDA broth was added, followed by 20 μL of MTT solution (1 g/L). After 4-12 h incubation at 30° C., reduced MTT was extracted with 1 mL of acidic isopropyl alcohol (1 mL of 12 N HCL in 100 mL IPA) The solution containing the dissolved colored formazan was centrifuged and the A_{570 nm} of the supernatant was determined in a UV-VIS spectrophotometer (Perkin-Elmer).

Fluorescent Probe Staining

FungaLight™ (Molecular Probes, Eugene Oreg.), a fluorescent live/dead assay, was used as described by the manufacturer. Stained samples (100 μL) were applied to clean, L-lysine coated (Sigma, St. Louis Mo.) glass slides, air dried, and washed with sterile double distilled water. The slides were observed using a Leica inverted microscope (Leica, Wetzlar Germany) equipped with DIC and a multiple fluorescent filter turret. In instances where the results were quantitated, images from 10 random fields were obtained using both DIC and epi-fluorescence. The number of viable and non-viable A. niger mycelia or spores were counted and tabulated as described in the text.

Post-Polymerization Quaternization

Alkyl chain effect for biocidal activity. To prepare PQA brushes carrying various hydrophilicities, quaternization was carried out using different carbon numbers of alkyl bromide. Contact angle with water of treated PQA brushes with different alkyl bromide was measured and the results were shown in Table 3. The contact angles of PQA brush treated with longer alkyl chain such nonyl (C9) and dodecyl (C12) were showing larger than with shorter alkyl chain such ethyl and hexyl, that is, treated PQA brushes with C9 and C12 showed highly hydrophobic property on the surface. The structure of the PQA brushes treated different alkyl bromide on the silicon wafer was characterized by transmittance FT-IR.

TABLE 3

| | | | | Biocidal effect | |
|---|---|---|---|---|---|
| | Estimated | Contact | Bindable QA | | Number |
| | conversion | angle | unit | | redered |
| Cn | (%) | (°) | (×10¹⁵ N⁺/cm²) | challenge | harmless |
| 2 | 97 | 15 | 14.36 | 3.77 E7 | 3.64 E7 (98%) |
| 6 | 91 | 55 | 17.59 | 3.77 E7 | 3.77 E7 (100%) |
| 9 | 61 | 80 | 13.72 | ? | ? |
| 12 | 39 | 95 | 6.79 | 3.77 E7 | 1.86 E7 (49%) |

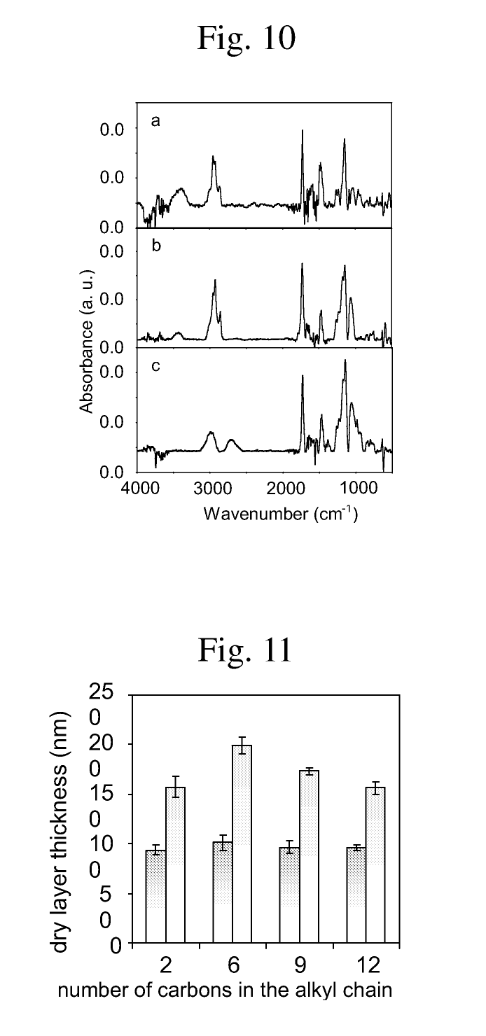

FIG. 10 illustrates transmittance FTIR spectra obtained from PQA brushes quaternized with 1-bromohexane (a), with 1-bromononane (b) and with 1-bromododecane (c) on the double side polished silicon wafer. As shown in FIGS. 10(a) and 10(b), larger absorption bands at 2965 and 2928 cm⁻¹, which could be assigned to asymmetric and symmetric stretches of the methylene chain of hexyl or nonyl group, can be observed and the absorption bands at 2820 and 2770 cm⁻¹ of the N—(CH₃)₂ are not present. However, after quaternization with 1-bromododecane, broad absorption bands at 2990 and 2770 cm⁻¹ could be assigned to the alkyl chain of dodecyl and the unreacted dimethylamine group, respectively.

For further analysis, the layer thicknesses of the polymer brushes after reaction with alkyl bromide having different carbon chain were measured. In all cases we started with PDMAEMA brush with a thickness of approximately 100 nm. Upon quaternization, an increasing ratio (L₂/L₁) of thickness was observe, with the ratio depending on the alkylbromide used. For example, we observed a large increase in the layer thickness to 198 nm in the case of quaternization with 1-bromohexane (C6). Although a larger increase in molecular weight occurs after quaternization with 1-bromododecane (M₂/M₁=2.59), a smaller change in layer thickness was observed for 1-bromododecane (L₂/L₁=1.62) as shown in FIG. 11. Murata and Rühe had reported that estimation of degree of PEGylation to the active ester polymer brushes by comparison with the changes in between the layer thickness and molecular weight of repeating unit of polymer chain before and after the PEGylation using surface plasmon spectroscopy. Murata, H.; Oswald Prucker, O.; Rühe, J., Synthesis of Functionalized Polymer Monolayers from Active Ester Brushes, Macromolecules 2007, 40, 5497-5503. In the studies of the present invention, we considered conversion of quaternization (f) to the polymer side chain with eq (3) set forth below. The f was inserted into eq (3) and density of the brushes before and after quaternization was assumed a nearly constant (ρ_{PDMAEMA}≈ρ_{PQACn}).

As set forth above, layer thicknesses of the PQA polymer brushes had increased after quaternization with alkyl bromides. To further understand this behavior, we consider that the quaternization reaction leads to increasing in the molecular mass of the attached polymers. This must also lead to differences in the thickness of the layer before and after quaternization. The thickness L of a polymer monolayer is given by:

$$L = \frac{Γ \cdot \overline{M} n}{ρ \cdot {N}_{A}} \qquad ( 1 )$$

with Γ being the graft density of the polymer chains, M̅n their number average molecular weight, ρ their density and Avogadro number N_{A}. Now, the ratio (L₂/L₁) of the layer thickness before (1) and after (2) reaction with an alkyl bromide can be expressed as follows,

$$\frac{{L}_{2}}{{L}_{1}} = \frac{{Γ}_{2} \cdot {\overline{M}}_{n 2} \cdot {ρ}_{1}}{{Γ}_{1} \cdot {\overline{M}}_{n 1} \cdot {ρ}_{2}} \qquad ( 2 )$$

Assuming that the quaternization with bromoethane proceeds quantitatively and does not change the graft density of the layers (Γ₁=Γ₂), we can re-write eq (2) as follows

$$\frac{{L}_{2}}{{L}_{1}} = \frac{{M}_{2} \cdot {ρ}_{1}}{{M}_{1} \cdot {ρ}_{2}} \qquad ( 3 )$$

Here M₁ and M₂ are the molar masses of the repeat units before (1) and after quaternization (2). If we assume a close density of the brushes (ρ₁≈ρ₂), we find that L₂/L₁ is equal to the ratio of the molar masses of the units before and after reaction (M₂/M₁). In the light of this calculation, the observed behavior for the quaternization of the PDMAEMA brushes with bromoethane becomes clear. Since the molar mass of repeat unit PQA brush (Mw=266.2 g/mol) is larger than that of PDMAEMA brush (Mw=157.2 g/mol), therefore, the layer thickness after quaternization is increased (L₂>L₁).

$$\frac{{L}_{PQACn}}{{L}_{PDMAEMA}} = \frac{f \cdot {M}_{PQACn} + ( 1 - f ) \cdot {M}_{DMAEMA}}{{M}_{DMAEMA}} \qquad ( 4 )$$

since M_{PQACn} is shown as M_{DMAEMA}+M_{Cn}, eq (4) was re-written as follow

$$\frac{{L}_{PQACn}}{{L}_{DMAEMA}} = \frac{{M}_{DMAEMA} + f \cdot {M}_{Cn}}{{M}_{DMAEMA}} \qquad ( 5 )$$

Here M_{Cn} is molecular weight of used alkyl bromides. Then, the f can be shown as eq (6).

$$f = \frac{{M}_{DMAEMA}}{{M}_{Cn}} \cdot ( \frac{{L}_{PQACn}}{{L}_{DMAEMA}} - 1 ) \qquad ( 6 )$$

Conversion of quaternization can be estimated using eq (6) as summarized in Table 3. In the cases of quaternization with 1-bromononane and 1-bromododecane, the conversion was found to decrease (compared to quaternization with shorter chain alkyl bromides) to 61 and 39%, respectively. Without limitation to any mechanism, it is possible that larger alkyl bromide could not reach amino group on the polymer chain as a result of interference with other alkyl chains in the structure as the reaction progressed, and that the diffusion into the dense polymer brush is relatively low. Fluorescein staining test of these PQA brushes was carried out. The value of surface density of QA unit of treated brushes as shown in Table 3 is also supporting the reduction of quaternization yield with longer chain alkyl bromides.

The foregoing description and accompanying drawings set forth the preferred embodiments of the invention at the present time. Various modifications, additions and alternative designs will, of course, become apparent to those skilled in the art in light of the foregoing teachings without departing from the scope of the invention. The scope of the invention is indicated by the following claims rather than by the foregoing description. All changes and variations that fall within the meaning and range of equivalency of the claims are to be embraced within their scope.

What is claimed is:

1. A method of applying a biocidal polymer to a surface comprising:
applying a solution of the biocidal polymer to the surface, wherein the biocidal polymer comprises biocidal groups and the biocidal polymer is insoluble in water.

2. The method of claim 1 wherein the biocidal groups are selected from the group consisting of quaternary salt groups or haloamine groups.

3. The method of claim 1 wherein the biocidal polymer is formed via a process comprising a radical polymerization.

4. The method of claim 2 wherein the biocidal polymer is formed via a process comprising a controlled radical polymerization.

5. The method of claim 4 wherein the quaternary salt groups comprise at least one of quaternary ammonium salt groups and quaternary phosphonium salt groups.

6. The method of claim 5 wherein the quaternary salt groups are quaternary ammonium salt groups.

7. The method of claim 6 wherein the biocidal polymer is formed from radically polymerizable monomers including at least one amino group.

8. The method of claim 7 wherein the radically polymerizable monomers comprise at least one of 2-(dimethylamino)ethyl methacrylate), 4-vinyl pyridine, 2-vinyl pyridine, N-substituted acrylamides, N-acryloyl pyrrolidine, N-acryloyl piperidine, acryl-L-amino acid amides, acrylonitriles, methacrylonitriles vinyl acetates, 2-hydroxy ethyl methacrylate, p-chloromethyl styrene, and derivatives and substituted varieties of such monomers.

9. The method of claim 6 wherein the quaternary ammonium salt groups are formed by reaction of a compound of the formula R₈Q with amino groups on radically polymerizable monomers reacted to form the polymer or with amino groups on a precursor polymer formed by polymerization of radically polymerizable monomers, wherein R₈ is an alkyl group of at least 8 carbon atoms, a fluorinated alkyl group of at least 8 carbon atoms or an aromatic group and Q is a halide, CF₃SO₃ or CF₃CO₂.

10. The method of claim 9 wherein the quaternary ammonium salt groups are formed by reaction of a compound of the formula R₈Q with amino groups on radically polymerizable monomers reacted to form the biocidal polymer.

11. The method of claim 9 wherein R₈ is an alkyl group or a fluorinated alkyl group.

12. The method of claim 9 wherein R₈ is an alkyl group or a fluorinated alkyl group of at least 8 carbon atoms.

13. The method of claim 9 wherein R₈ is an alkyl group or a fluorinated alkyl group of at least 12 carbon atoms.

14. The method of claim 9 wherein the alkyl group is a C₈ to C₂₂ alkyl group.

15. The method claim 6, wherein the biocidal polymer comprises a repeat unit selected from the following formulae: 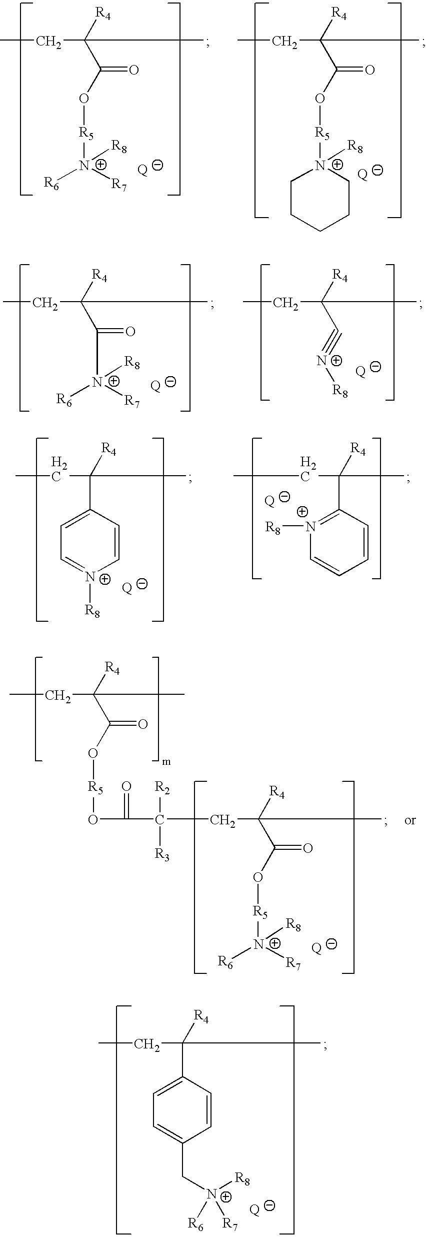
wherein R₂ and R₃ are, independently, H, CH₃, OOCC₂H₅ or CN, R₄ is H, CH₃, Cl or CN, R₅ is —(CH₂)ₙ— or —CH₂C(CH₃)₂CH₂—, wherein n is an integer from 1 to 6, R₆ and R₇ are, independently, a C₁-C₅ alkyl group, R₈ is an alkyl group of at least 8 carbon atoms, a fluorinated alkyl group of at least 8 carbon atoms or an aromatic group and Q is one of F, Cl, Br, I, CF₃SO₃ and CF₃CO₂.

16. The method of claim 1 wherein the polymer is hydrophobic.

17. A biocidal article formed by applying a solution of a biocidal polymer in a nonaqueous solvent to a surface, wherein the biocidal polymer comprises biocidal groups and the polymer is insoluble in water.

18. A polymer that is the reaction product of a radical polymerization of monomers comprising at least one radically polymerizable monomer of the following formulae: 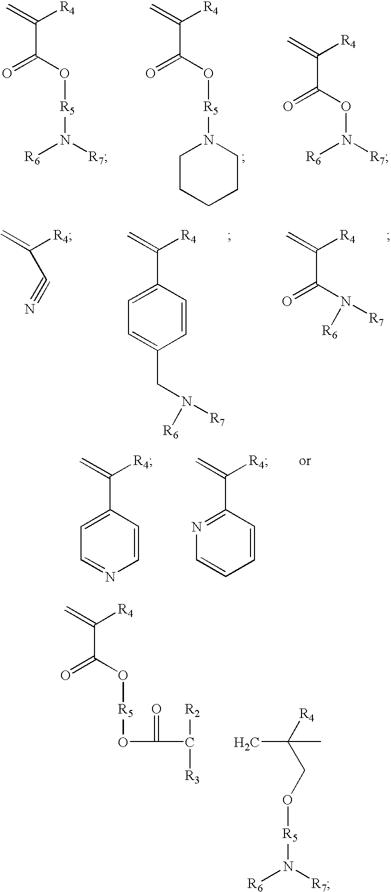
wherein an amine group of the radically polymerizable monomer is converted to a quaternary salt either before or after the radical polymerization by reaction with a compound of the formula R₈Q, wherein R₈ is an alkyl group of at least 8 carbon atoms, a fluorinated alkyl group of at least 8 carbon atoms or an aromatic group and Q is F, Cl, Br, I, CF₃SO₃ or CF₃CO₂, and wherein R₂ and R₃ are, independently, H, CH₃, OOCC₂H₅ or CN, R₄ is H, CH₃, Cl or CN, R₅ is —(CH₂)ₙ— or —CH₂C(CH₃)₂CH₂—, wherein n is an integer from 1 to 6, R₆ and R₇ are, independently, a C₁-C₅ alkyl group, such that the polymer is insoluble in water.

19. The polymer of claim 18 wherein the radical polymerization of monomers comprise at least one radically polymerizable monomer of the following formulae:

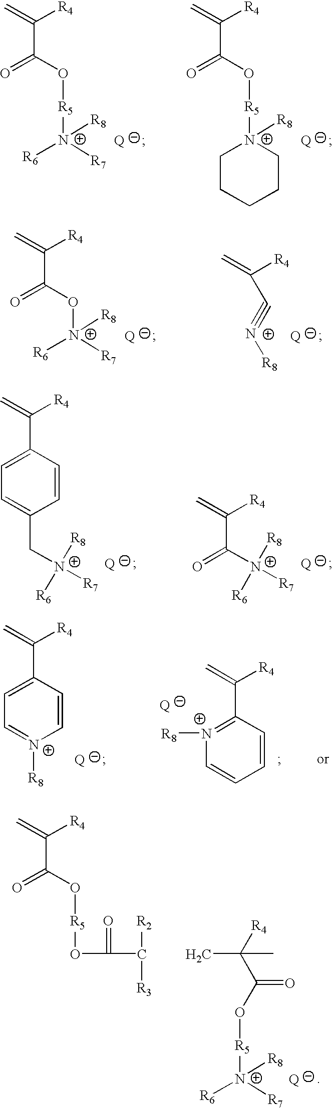

20. The polymer of claim 18 wherein the polymer is a homopolymer of at least one of the radically polymerizable monomers.

21. The polymer of claim 18 wherein the radical polymerization is a controlled radical polymerization.

22. The polymer of claim 21 wherein R₈ is an alkyl group or a fluoroalkyl group and Q is F, Cl, Br, or I.

23. The polymer of claim 22 wherein the alkyl group or the fluoroalkyl has at least 12 carbon atoms.

24. The polymer of claim 18 wherein the radically polymerizable monomer is.

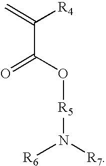

25. The polymer of claim 24 wherein R₈Q is 1-bromododecane.

26. A compound having the formula: 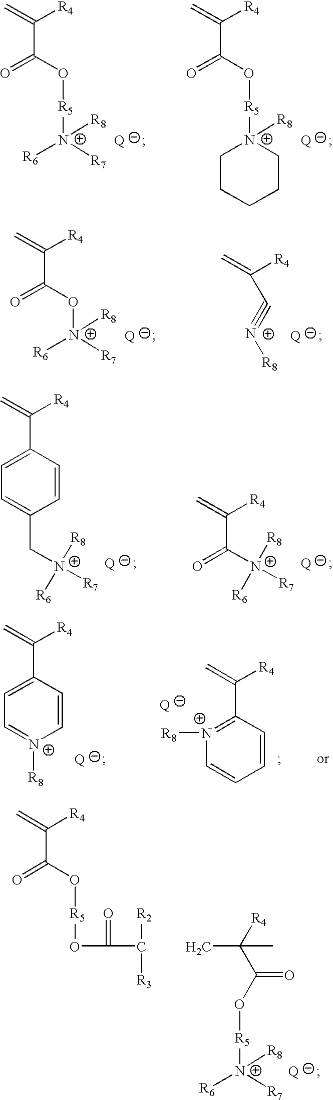
wherein R₂ and R₃ are, independently, H, CH₃, OOCC₂H₅ or CN, R₄ is H, CH₃, Cl or CN, R₅ is —(CH₂)ₙ— or —CH₂C(CH₃)₂CH₂—, n is an integer from 1 to 6, R₆ and R₇ are, independently, C₁-C₅ alkyl or isopropyl, R₈ is an alkyl group, a fluorinated alkyl group or an aromatic group; and Q is one of F, Cl, Br, I, CF₃SO₃ and CF₃CO₂.

27. The compound of claim 26 wherein R₈ is an alkyl group or at least 8 carbon atoms, a fluorinated alkyl group or at least 8 carbon groups or an aromatic group
